(19) United States
(12) Patent Application Publication
(10) Pub. No.: US 2022/0405680 A1
(43) Pub. Date: Dec. 22, 2022

(54) Automated Healthcare Provider Quality Reporting System (PQRS)

(71) Applicant: Modernizing Medicine, Inc., Boca Raton, FL (US)

(72) Inventors: Daniel Cane, Boynton Beach, FL (US); Michael Sherling, Palm Beach Gardens, FL (US)

(73) Assignee: Modernizing Medicine, Inc., Boca Raton, FL (US)

(21) Appl. No.: 17/730,900

(22) Filed: Apr. 27, 2022

Related U.S. Application Data
(63) Continuation of application No. 16/939,324, filed on Jul. 27, 2020, now abandoned.
Application No. 16/939,324 is a continuation of application No. 16/133,924, filed on Sep. 18, 2018, now abandoned.
Application No. 16/133,924 is a continuation of application No. 14/808,890, filed on Jul. 24, 2015, now abandoned.
(60) Provisional application No. 62/029,153, filed on Jul. 25, 2014.

(51) Int. Cl. G06Q 10/06; G16Z 99/00; G16H 40/20; G16H 10/60

(52) U.S. Cl. CPC G06Q 10/06395; G16Z 99/00; G16H 40/20; G16H 10/60

(57) ABSTRACT

An automated system for making quality measure submissions. In one embodiment, the system includes: a data input system; a data output system; a database of descriptive data items; a processor in communication with the data input system, the data output system and the database, and comprising a rules engine to traverse a hierarchical tree of denominator and numerator questions, using patient input and data items from the database of descriptive data items. In one embodiment, the invention relates to an automated review system for providing a multiple measure review of care including: a provider input device for inputting patient data; a patient database; a rules engine in communication with the patient database and traversing a plurality of denominator and numerator rules, using provider input patient data and patient data from the database to generate, from multiple encounters and multiple measures, the review of care subsequent to each visit.

### RELATED APPLICATIONS

This application is a continuation of U.S. patent application Ser. No. 16/939,324, filed on Jul. 27, 2020, which is a continuation of U.S. patent application Ser. No. 16/133,924, filed on Sep. 18, 2018, which is a continuation of U.S. patent application Ser. No. 14/808,890, filed on Jul. 24, 2015, which claims priority to and the benefit of U.S. Provisional Application No. 62/029,153, filed on Jul. 25, 2014, the entire disclosure of which is incorporated by reference herein.

### FIELD OF THE INVENTION

The invention relates generally to reporting systems for compliance with government regulations and more specifically to automated records management systems for physician or other healthcare provider reporting.

### BACKGROUND OF THE INVENTION

Various government regulations in various jurisdictions require that providers report on various aspects of patient treatment. For example in the United States, the Centers for Medicare and Medicaid Services (CMS) requires providers to report various measurements to CMS for qualifying patients treated during a calendar year.

In the United States, the CMS regulations require “quality measures” that include a numerator and a denominator that permit the calculation of the percentage of a defined patient population that receive a particular process of care or achieve a particular outcome. The denominator population is defined by: demographic information; certain International Classification of Diseases, Ninth Revision; Clinical Modification (ICD-9-CM) diagnosis (Jan. 1, 2014-Sept. 30, 2014); International Classification of Diseases, Tenth Revision; Clinical Modification (ICD-10-CM) diagnosis (Oct. 1, 2014-Dec. 31, 2014); Current Procedural Terminology (CPT); and Healthcare Common Procedure Coding System (HCPCS) codes specified in the particular measurement that are submitted by individual eligible professionals as part of a claim for covered services under the Physician Fee Schedule (PFS) for claims-based reporting.

If a given patient is within the defined patient population (the denominator), then the applicable Quality Data Codes or QDCs (for example CPT Category II codes or G-codes) that define the numerator must be submitted to satisfactorily report quality data for a given quality measure for claims-based reporting. The quality measure specifications also define circumstances in which a patient may be appropriately excluded for example: for CPT Category II code modifiers such as 1P, 2P and 3P; if Quality Data Codes are not available; if the provider describes a medical, patient, system or other reason for performance exclusion. When such performance exclusion does not apply, a quality measure-specific CPT Category II reporting modifier 8P or Quality Data Codes must be used to indicate if the standard of care was not provided for a reason not otherwise specified. Thus, such reporting of each quality measure specifies detailed reporting information. Although various government regulators may or may not utilize these same QDCs, the use of clinical concepts described for each quality measure in the numerator are generally required when submitting a report to a regulating body or its designee, which in one embodiment is a PQRS registry.

Generally, if a patient meets certain inclusion criteria (the denominator), then the provider needs to determine if the patient met or did not meet certain goals, or that the care provided did or did not perform certain activities (the numerator). Further, these calculations need to happen for each patient encounter for the defined patient populations. The set of rules of inclusion/exclusion and the specificity of reporting make the calculation logic very difficult to understand, difficult for a provider to follow, and very time-consuming. Such reporting typically involves certain simple inclusion criteria such as, is the patient Medicare Part B eligible and over the age of 18, followed by a massive set of reporting logic decisions involving massive lists of ICD, CPT, and other treatment/outcome criteria. The reporting logic can range from simply “the patient must have the following ICD-10 code on their encounter” to much more complex requirements such as “must contain all of the following codes and at least one of the following codes, and can never contain yet another code.” A sample quality measure specification used by the United States is shown in FIG. 1 (a-c). In the United States, there are over 300 measures on which providers can report. The instructions for calculation are over 600 pages long.

Currently available reporting systems generally use a series of “yes” and “no” questions to determine if a patient meets eligibility and what the numerator should be. For this to work properly, the providers must know which quality measures are going to be collected and what the requirements for the measures are. The provider is then asked a series of questions to determine if the patient meets all the reporting criteria and the details of the provider's examination, impression, plans, and patient activities. An example of such a questionnaire is shown in FIG. 2. Again, even with such reporting aids, quality measure reporting is difficult to comply with and very time consuming.

The present invention addresses these issues.

### SUMMARY OF THE INVENTION

In one aspect, the invention relates to an automated system for making quality measure submissions. In one embodiment, the system includes: a data input system; a data output system; a database of descriptive data items; a processor in communication with the data input system, the data output system and the database, and comprising a rules engine constructed to traverse a hierarchical tree of denominator and numerator questions using data input that describes the patient and data items from the database of descriptive data items. In another embodiment, descriptive data items comprise SNOMED, ICD9, ICD10, RxNorm, LOINC, CPT and Metathesaurus code value pairs. In yet another embodiment, descriptive data items comprise a descriptive string and at least one of a code system and a specific code value. In still another embodiment, the descriptive data items are linkable to other descriptive data items. In still yet another embodiment, the system applies a descriptive data item to a specific object input by a user.

In one embodiment, the user associates a fact with the specific object and the system assigns a descriptive data item to the fact associated with the specific object. In another embodiment, the system determines whether the descriptive data item assigned to the fact should be assigned as metadata. In yet another embodiment, the rules engine examines the facts associated with an object and applies a hierarchical tree of rules to metadata assigned to the facts to determine if the facts meet the requirements of the rules. In still another embodiment, the rules engine uses the metadata assigned to the facts to determine an outcome value of a specific rule. In still yet another embodiment, the rules engine associates the outcome with the object to which the fact applies.

In one embodiment, the rules engine determines an outcome value of a specific rule in response to an outcome value generated by other rules. In another embodiment, the rules engine determines an outcome value of a specific rule in real-time to output the outcome value to the user. In yet another embodiment, the system generates an output report in response to all objects for which the user has associated facts.

In another aspect, the invention relates to an automated review system for providing a multiple measure review of patient care. In one embodiment, the automated review system includes a provider input device for inputting patient data; a patient database; and a rules engine in communication with the patient database and traversing a plurality of denominator and numerator rules, using both provider input patient data and patient data from the patient database to generate, from multiple encounters and multiple measures, the review of patient care subsequent to each patient visit. In another embodiment, the review is automatically provided to a PARS registry.

### BRIEF DESCRIPTION OF THE DRAWINGS

The structure and function of the invention can be best understood from the description herein in conjunction with the accompanying figures. The figures are not necessarily to scale, emphasis instead generally being placed upon illustrative principles. The figures are to be considered illustrative in all aspects and are not intended to limit the invention, the scope of which is defined only by the claims.

FIGS. 1(a-c) is an example of the specification of a quality measure known to the prior art;

FIG. 2 is an example of a query input form;

FIG. 3 is an embodiment of a system constructed in accordance with the invention;

FIG. 4(a-c) is an embodiment of a data structure of a quality measure in accordance with the invention;

FIG. 5 is an embodiment of a data structure of the logic block in accordance with the invention;

FIG. 6 is a flow diagram of an embodiment of the steps in a patient visit relating to tobacco use;

FIG. 7 is a flow diagram of an embodiment of the steps in assigning codes to the patient visit shown in FIG. 6;

FIG. 8 is an embodiment of a display screen in accordance with the invention;

FIG. 9 is an embodiment of a detail screen in accordance with the invention; and

FIG. 10 is an embodiment of a report constructed in accordance with the invention.

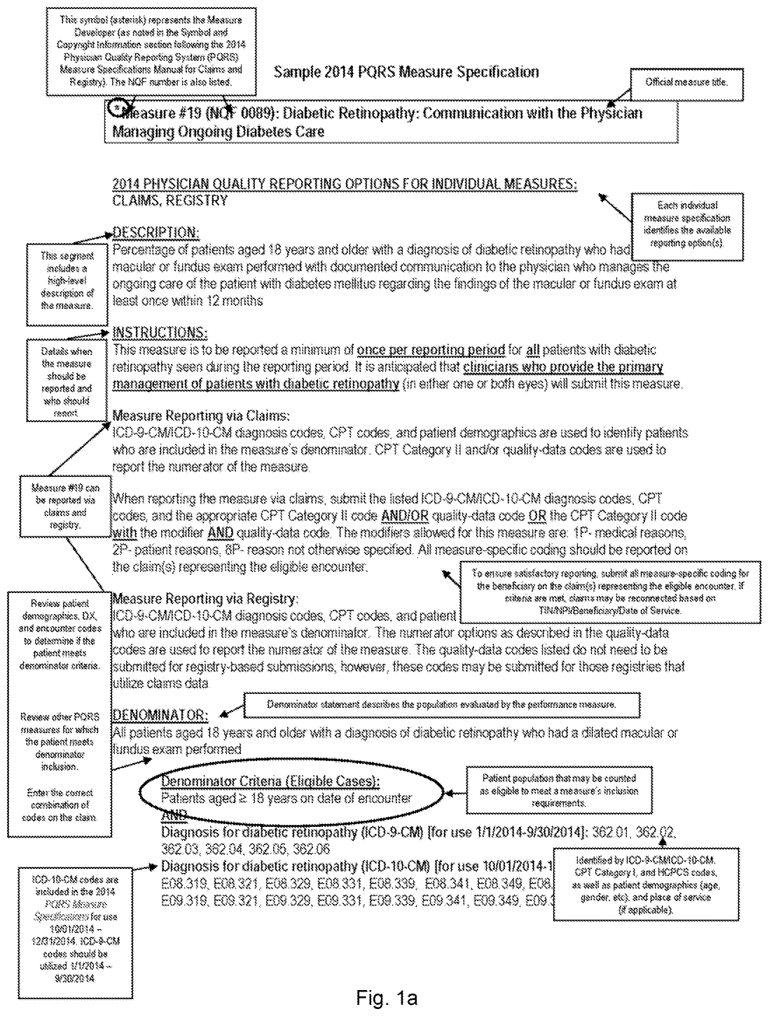

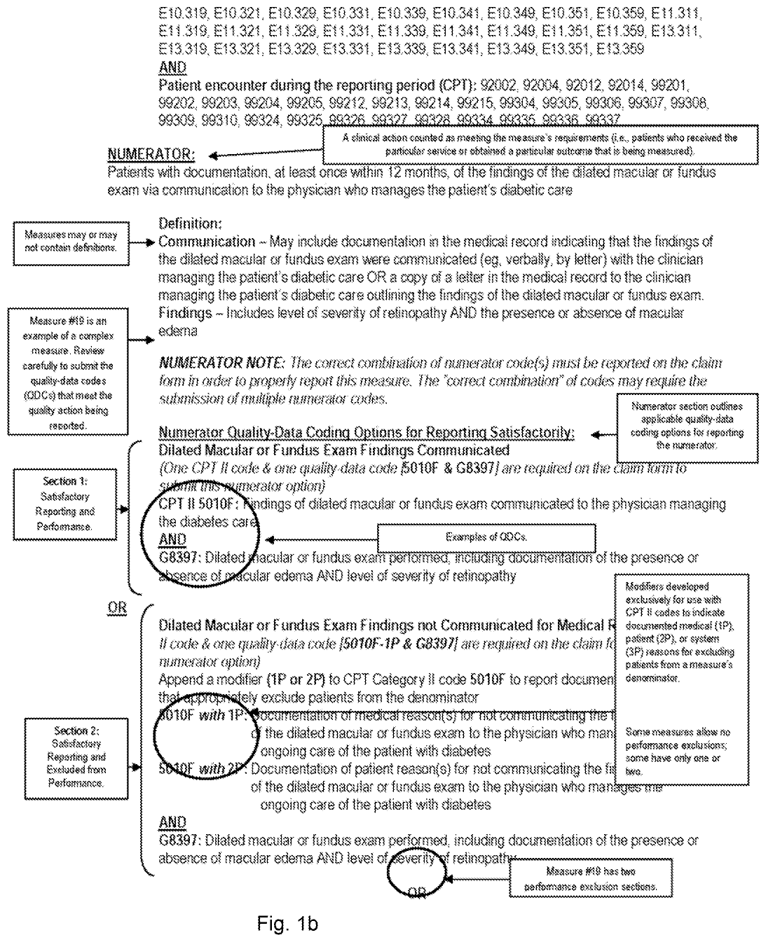

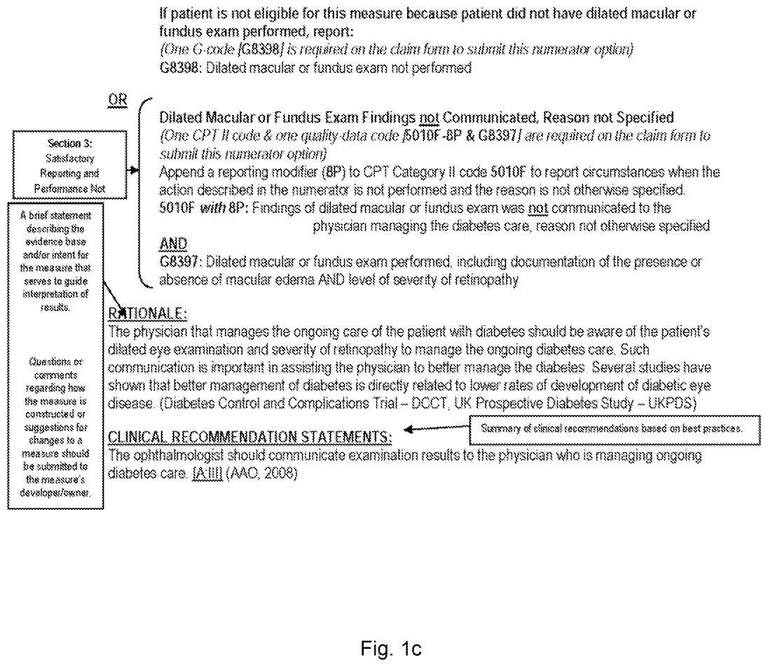

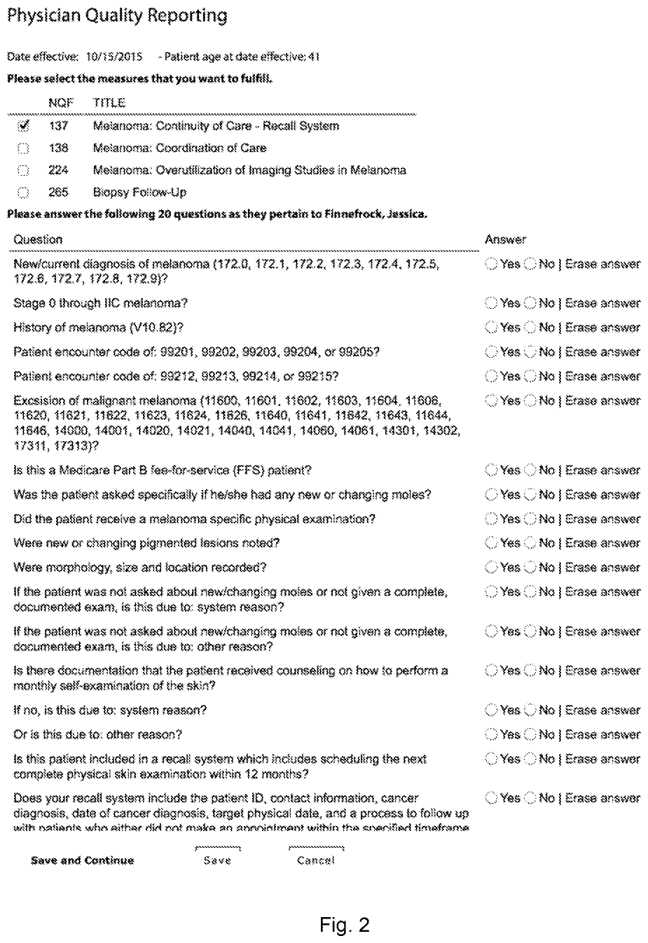

### DESCRIPTION OF A PREFERRED EMBODIMENT

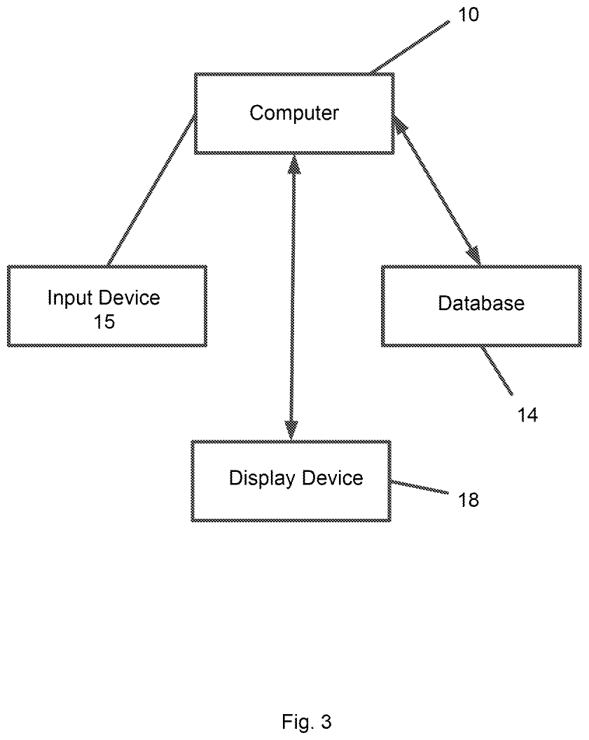

In brief overview and referring to FIG. 3, a system constructed in accordance with the present invention includes a computer or processor 10 having a rules engine in communication with a records management database 14, an input device 15, and an output display 18, which may generate printed reports. Patient encounters are input to the system through the input device 14. To generate a report, the processor 10 executes the rules of the rules engine that accesses the patient database 14 to generate a report through the output device 18.

### Structure

The present invention is based on structured descriptive data items. Such data includes but is not limited to past medical history, current medications, exam findings, diagnoses, treatments, lab results, and severity assessments. Descriptive data items may be linked together with other descriptive data items. Each data item can be further “tagged” with additional codes. A non-limiting list of standards for the descriptive data items which the system can tag with additional metadata is shown in Table 1.

TABLE 1

| | |
|---|---|
| Past Medical History | SNOMED |
| Social History | SNOMED |
| Problem List | SNOMED, ICD9/10 |
| Medications, Prescriptions | RxNorm, SNOMED |
| Lab Orders/Results | LOINC, SNOMED |
| HPI/Chief Complaint | SNOMED, LOINC, Metathesaurus |
| Diagnoses | ICD9/10, SNOMED, Metathesaurus |
| Morphologies | SNOMED |
| Exam | SNOMED, LOINC, Metathesaurus |
| Procedures | SNOMED, LOINC, Metathesaurus |

These descriptive data items include, but are not limited to: the general standard elements, the Systemized NOmenclature of MEDicine (SNOMED) which includes codes for medical history, social history, problem list, medications and prescriptions, laboratory orders and laboratory results, history of present illness (HPI) and chief complaint, diagnoses, morphologies, examinations and procedures; the laboratory standard, Local Observation Identifier for Names and Codes (LOINC) which includes laboratory orders and laboratory results, history of present illness (HPI) and chief complaint, examinations and procedures; the catalog of standard drug and drug delivery device names (RxNORM) for medications and prescriptions; the International Classification of Disease (ICD(n)) where (n) is the number of the present edition, which describes the problem list and diagnoses; and the Metathesaurus, a multi-lingual vocabulary database that provides alternative names to the same concepts for history of present illness (HPI) and chief complaint, diagnoses, examinations and procedures.

In one embodiment, the system uses an XML-based markup language to create the structured data system. However, other languages and schema may be used.

An example of a rule is shown for a foot examination:

<mm:examBullet examElement=“foot inspection, sensation by monofilament, pedal

pulses palpated” renderElementSelf=“true” isBodyLocation=“true” tabLabel=“Diabetic Foot”>

<mm:descriptiveCoding>

<mm:descriptiveCodingItem codeSystem=“SNOMED”

codeValue=“164480009” codeName=“On examination - foot (finding)”/>

<mm:descriptiveCodingItem codeSystem=“SNOMED”

codeValue=“134388005” codeName=“Monofilament foot sensation test”/>

<mm:descriptiveCodingItem codeSystem=“SNOMED”

codeValue=“91161007” codeName=“Pedal pulse taking”/>

</mm:descriptiveCoding>

</mm:examBullet>

In this example, the user enters data or facts (such as age, body temperature, etc.) about a specific object (such as a patient, body part, etc.) into the system relating to a specific exam performed. The system associates those facts and relative descriptive data items with the specific object. The rules engine examines all the facts associated with a specific object and determines if the fact associated with the object matches with any of the descriptive data items that are present in the database. If so, the engine then determines if those matching data items should be applied to the fact as metadata about the fact. The rules engine then includes the metadata tags for the appropriate codes.

The rules engine examines a set of facts about an object, and while examining those facts, applies a hierarchical tree of rules to the metadata tags added to those facts to determine if the set of facts meets the requirements of any of the defined rules. If a set of facts matches the rules, the rules engine uses the metadata attached to those facts to determine the outcome value of the specific rule. This outcome value is associated to the object to which the fact applies. Also, rules can generate an output value based on the outcome of other rules. Reports may be generated in real time by applying rules to objects and the facts associated with the objects.

In a second example, the results of the administration of a drug during the encounter are included. In this example, a flu vaccine was recommended but the patient declined, instead opting to have the vaccine administered at a later time and location:

<mm:var name=“influenzaImmunization” type=“select” label=“PQRS 110: Preventive

Care and Screening: Influenza Immunization” stickyValues=“false” tabLabel=“Details”>

<mm:varOption value=“n/a” isDefault=“true”/>
<mm:varOption value=“Influenza Immunization Administered during Influenza

season”>

<mm:descriptiveCoding>

<mm:descriptiveCodingItem codeSystem=“Metathesaurus”

codeValue=“C2959021” codeName=“PATIENT DOCUMENTED TO HAVE RECEIVED INFLUENZA
VACCINATION DURING INFLUENZA SEASON”/>

</mm:descriptiveCoding>

</mm:varOption>
<mm:varOption value=“Influenza Immunization previously received during

influenza season”>

<mm:descriptiveCoding>

<mm:descriptiveCodingItem codeSystem=“Metathesaurus”

codeValue=“C2959021” codeName=“PATIENT DOCUMENTED TO HAVE RECEIVED INFLUENZA
VACCINATION DURING INFLUENZA SEASON”/>

</mm:descriptiveCoding>

</mm:varOption>
<mm:varOption value=“Influenza Immunization not Administered for

Documented Reasons.” >

<mm:descriptiveCoding>

<mm:descriptiveCodingItem codeSystem=“Metathesaurus”

codeValue=“C1718261” codeName=“Reason influenza virus vaccine not received”/>

</mm:descriptiveCoding>

</mm:varOption>
<mm:varOption value=“Influenza Immunization Ordered or Recommended, but

not Administered” >

<mm:descriptiveCoding>

<mm:descriptiveCodingItem codeSystem=“Metathesaurus”

codeValue=“C3248434” codeName=“INFLUENZA IMMUNIZATION ORDERED OR
RECOMMENDED (TO BE GIVEN AT ALTERNATE LOCATION OR ALTERNATE PROVIDER); VACCINE
NOT AVAILABLE AT TIME OF VISIT”/>

</mm:descriptiveCoding>

</mm:varOption>
<mm:varOption value=“Influenza immunization was not ordered or

administered, reason not given”/>

</mm:var>

In this system, the provider is not entering any data he or she would not normally enter to document a patient encounter. That is, no additional questions are requested by the system. The system's rules automatically populate using the data in the patient database.

Any data item can be tagged. Further, because the structured data is extremely detailed, the system can differentiate the nuances in metadata tagging. For example, the system not only knows if a provider recorded a pain intensity level, but what the specific level was. This level of detail is required for an automated quality measure calculation to take place.

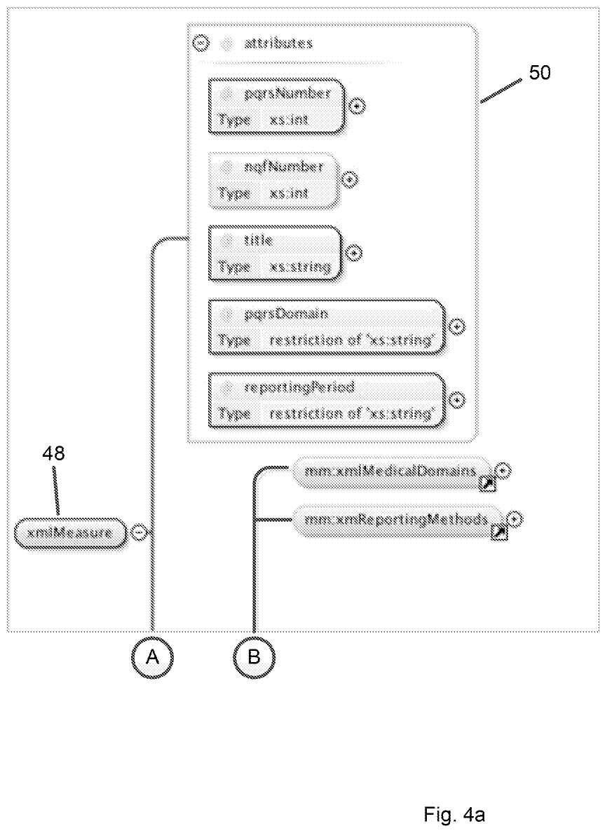

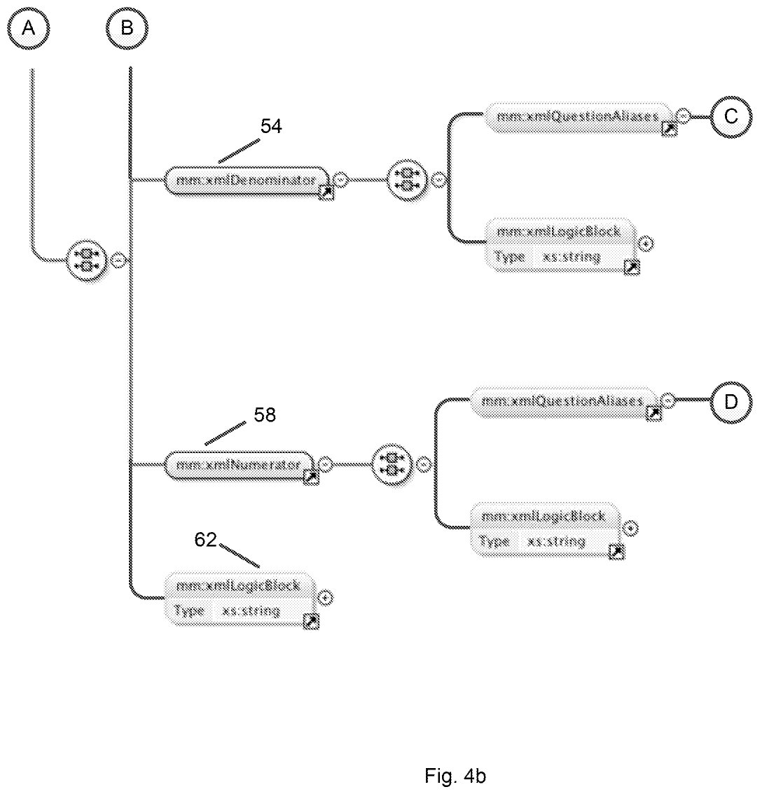

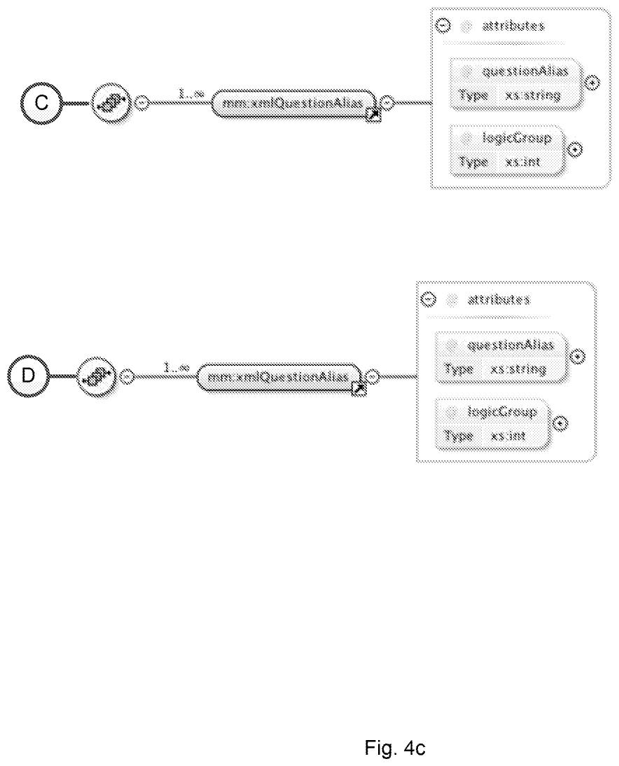

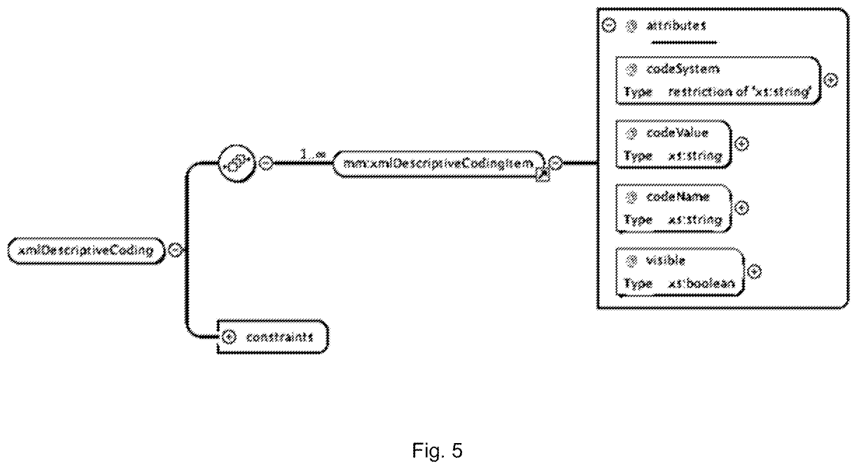

To perform the calculation, the system organizes each quality measure in terms of its numerator and denominator questions (FIG. 4). Each quality measure 48 includes a set of attributes 50 and a set of denominator 54 and numerator 58 questions in a hierarchical tree. The logic block 62 permits sets of logic to be used together. Thus, a single question will have logic to determine if it is true or false. The logic takes the form of either discrete calculated data or a coded logic “block.” In one embodiment, the block is coded in JavaScript. The calculated data permits the expression of the following types of logic: Find All, Find Any, Exclude All, Exclude Any. Each of these expressions behaves in a different way and can be used in combination to express the desired logic. Each of these expressions contains a 0-to-many list of descriptive codes (metadata) to compare with (FIG. 5). If the logic finds a match, it returns “true” and then logic block will branch to the follow-up “if true section” and process any follow up questions. The same is also the case with the “false” branch. At each question and follow-up question, CPT-codes and modifiers can be calculated and added to the overall quality measure. This format allows for logic to be chained together for computing all of the variability needed for the variety of quality measure calculations.

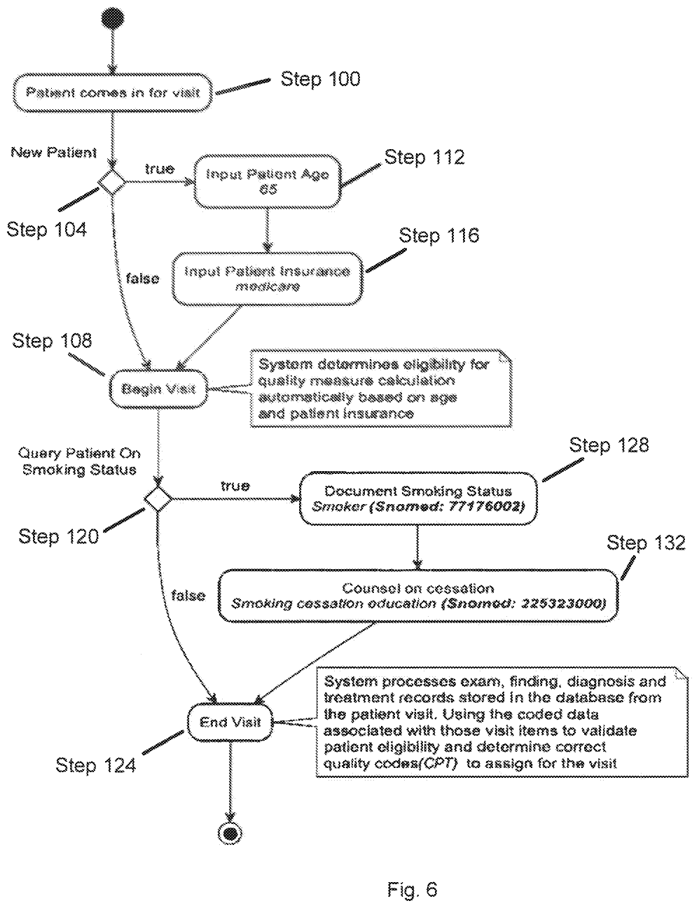

Considering an example in which a patient is counseled about smoking and referring to FIG. 6, the patient begins the visit (Step 1) and the user enters whether the patient is a new patient (Step 104). If the patient is not a new patient, the visit begins (Step 108). If the patient is a new patient initial data is collected about the patient such as age (Step 112) and insurance plan (Step 116), at which point the system determines whether a quality measure calculation will be performed on this patient based on age and insurance, at which point the visit begins (Step 108).

The patient is asked whether he or she is a smoker and if the answer is “no,” the remainder of the examination take place (Step 124). If the answer is “yes,” the fact is entered into the database as “smoker” and assigned the SNOMED code 77176002 by the system. The clinician then counsels the patient about the health effects of smoking and the system enters the SNOMED code for “smoking cessation education” 225323000 into the database and the examination proceeds.

Upon completion of the visit (Step 124), the system processes the examination findings, diagnosis, and treatment data records stored in the database, and uses the coded metadata to validate patient eligibility and determine the correct quality codes.

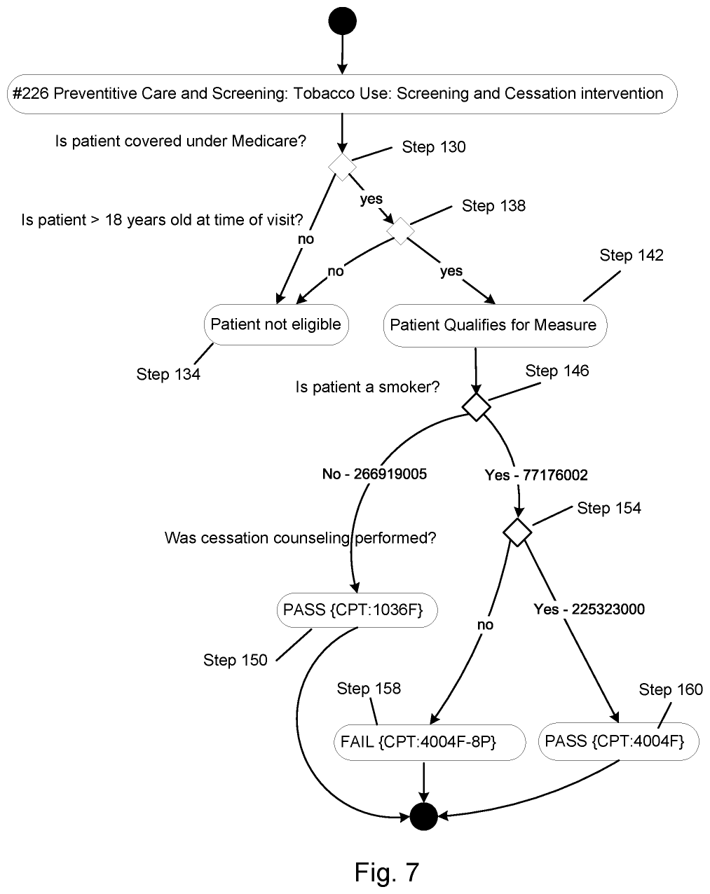

Referring to FIG. 7, the system determines the eligibility of the patient for a quality measure calculation by examining the data about the patient. The system first considers whether the patient is covered by Medicare (Step 130), if the answer is no the patient is determined to be ineligible (Step 134). If the patient is covered by Medicare, the system then determines if the patient is over 18 years old (Step 138). If the patient is not over 18 years old, the patient is again found to be ineligible (Step 134). If the patient is over 18 years old the patient is eligible for a quality measure calculation.

The system determines whether the patient is a smoker (Step 146) and if the answer is “no” the CPT value is set to “no” (Step 150). If the patient is a smoker, the system determines if the counseling was performed (Step 154). If the answer is “no,” CPT is set to “fail” and if the answer is “yes,” the CPT is set to “pass” step 160. The system will also generate information to the user as to why an entry is a “fail” and what can be done to correct it.

As an example, a quality measure for this scenario has the structure:

<xmlMeasure pqrsNumber=“226” title=“Preventive Care and Screening: Tobacco

Use: Screening and Cessation Intervention”
pqrsDomain=“COMMUNITY_POPULATION_HEALTH” reportingPeriod=“EACH_VISIT”>

<xmlDenominator>

<xmlQuestionAliases>

<xmlQuestionAlias questionAlias=“medicarePartB”/>
<xmlQuestionAlias questionAlias=“patientGreaterEqualThan18”/>
<xmlQuestionAlias questionAlias=“tobaccoByEncounter”/>

</xmlQuestionAliases>

</xmlDenominator>
<xmlNumerator>

<xmlQuestionAliases>

<xmlQuestionAlias questionAlias=“tobaccoScreenedSmokerNegative”/>

</xmlQuestionAliases>

</xmlNumerator>

</xmlMeasure>

with the denominator question having the structure:

<xmlQuestion questionAlias=“tobaccoByEncounter” questionText=“Is there an

encounter code?”>

<xmlLogicForTrue>

<xmlFindAny>

<xmlDescriptiveCoding>

<xmlDescriptiveCodingItem codeSystem=“CPT” codeValue=“90791”/>
<xmlDescriptiveCodingItem codeSystem=“CPT” codeValue=“90792”/>
<xmlDescriptiveCodingItem codeSystem=“CPT” codeValue=“90832”/>
<xmlDescriptiveCodingItem codeSystem=“CPT” codeValue=“90834”/>
<xmlDescriptiveCodingItem codeSystem=“CPT” codeValue=“90837”/>
<xmlDescriptiveCodingItem codeSystem=“CPT” codeValue=“90839”/>
<xmlDescriptiveCodingItem codeSystem=“CPT” codeValue=“90845”/>
<xmlDescriptiveCodingItem codeSystem=“CPT” codeValue=“92002”/>
<xmlDescriptiveCodingItem codeSystem=“CPT” codeValue=“92004”/>
<xmlDescriptiveCodingItem codeSystem=“CPT” codeValue=“92012”/>
<xmlDescriptiveCodingItem codeSystem=“CPT” codeValue=“92014”/>
<xmlDescriptiveCodingItem codeSystem=“CPT” codeValue=“96150”/>
<xmlDescriptiveCodingItem codeSystem=“CPT” codeValue=“96151”/>
<xmlDescriptiveCodingItem codeSystem=“CPT” codeValue=“96152”/>
<xmlDescriptiveCodingItem codeSystem=“CPT” codeValue=“97003”/>
<xmlDescriptiveCodingItem codeSystem=“CPT” codeValue=“97004”/>
<xmlDescriptiveCodingItem codeSystem=“CPT” codeValue=“99201”/>
<xmlDescriptiveCodingItem codeSystem=“CPT” codeValue=“99202”/>
<xmlDescriptiveCodingItem codeSystem=“CPT” codeValue=“99203”/>
<xmlDescriptiveCodingItem codeSystem=“CPT” codeValue=“99204”/>
<xmlDescriptiveCodingItem codeSystem=“CPT” codeValue=“99205”/>
<xmlDescriptiveCodingItem codeSystem=“CPT” codeValue=“99212”/>
<xmlDescriptiveCodingItem codeSystem=“CPT” codeValue=“99213”/>
<xmlDescriptiveCodingItem codeSystem=“CPT” codeValue=“99214”/>
<xmlDescriptiveCodingItem codeSystem=“CPT” codeValue=“99215”/>
<xmlDescriptiveCodingItem codeSystem=“CPT” codeValue=“99406”/>
<xmlDescriptiveCodingItem codeSystem=“CPT” codeValue=“99407”/>
<xmlDescriptiveCodingItem codeSystem=“CPT” codeValue=“G0438”/>
<xmlDescriptiveCodingItem codeSystem=“CPT” codeValue=“G0439”/>

</xmlDescriptiveCoding>

</xmlFindAny>

</xmlLogicForTrue>

</xmlQuestion>

and the numerator having the structure:

<xmlQuestion questionAlias=“tobaccoScreenedSmokerNegative”

questionText=“Was the patient screened for tobacco and is not a smoker?” cptCode=“1036F”
performance=“pass”>

<xmlLogicForTrue>

<xmlFindAny>

<xmlDescriptiveCoding>

<xmlDescriptiveCodingItem codeSystem=“SNOMED”

codeValue=“8517006” codeName=“Ex-smoker”/>

<xmlDescriptiveCodingItem codeSystem=“SNOMED”

codeValue=“266919005” codeName=“Never smoked tobacco”/>

<xmlDescriptiveCodingItem codeSystem=“SNOMED”

codeValue=“266927001” codeName=“Tobacco smoking consumption unknown”/>

</xmlDescriptiveCoding>

</xmlFindAny>

</xmlLogicForTrue>
<xmlFollowUpIfFalse>

<xmlQuestionAliases>

<xmlQuestionAlias questionAlias=“tobaccoScreenedSmokerPositive”/>

</xmlQuestionAliases>

</xmlFollowUpIfFalse>

</xmlQuestion>
<xmlQuestion questionAlias=“tobaccoScreenedSmokerPositive” questionText=“Was

the patient screened for tobacco and is a smoker?” cptCode=“4004F” performance=“pass”>

<xmlLogicForTrue>

<xmlFindAny>

<xmlDescriptiveCoding>

<xmlDescriptiveCodingItem codeSystem=“SNOMED”

codeValue=“77176002” codeName=“Smoker”/>

<xmlDescriptiveCodingItem codeSystem=“SNOMED”

codeValue=“428041000124106” codeName=“Occasional tobacco smoker”/>

<xmlDescriptiveCodingItem codeSystem=“SNOMED”

codeValue=“428071000124103” codeName=“Heavy tobacco smoker”/>

<xmlDescriptiveCodingItem codeSystem=“SNOMED”

codeValue=“428061000124105” codeName=“Light tobacco smoker”/>

</xmlDescriptiveCoding>

</xmlFindAny>
<xmlFindAll>

<xmlDescriptiveCoding>

<xmlDescriptiveCodingItem codeSystem=“SNOMED”

codeValue=“225323000” codeName=“Smoking cessation education”/>

</xmlDescriptiveCoding>

</xmlFindAll>

</xmlLogicForTrue>
<xmlFollowUpIfFalse>

<xmlQuestionAliases>

<xmlQuestionAlias questionAlias=“tobaccoNotScreenedMedicalReason”/>

</xmlQuestionAliases>

</xmlFollowUpIfFalse>

</xmlQuestion>

With this structure, as the provider is entering data, the system collects all of the tagged metadata appropriate for this patient and encounter, and uses it for the measure calculation.

In overview, using the above example, when the system computes measure 226 of the quality measures which relates to Tobacco Screening and Cessation, the system first examines the inclusion criteria for the quality measure. All denominator requirements in a logic group must be met for the patient to be included in the calculation. In this example, the system ensures that the patient is Medicare Part B, over the age of 18, and has an encounter (CPT) code for an exam. If the inclusion criteria are met, then the system computes the numerator.

In this example, the numerator logic begins by looking for SNOMED codes that would indicate if the patient was screened for tobacco use or was not a smoker. If the system finds the presence of any of the SNOMED codes included in the Find Any logic block, then the system will return the corresponding CPT (in this case, code 1036F). There are no follow-up questions defined if the logic returns “true,” and in that case, the measure calculation is complete. If the logic returns “false” (meaning none of the SNOMED codes were found), the system will execute the question logic found in the follow-up “false” block. In this case, it would look for an alternative set of SNOMED codes and return the appropriate CPT codes and modifiers down the chain.

### Results

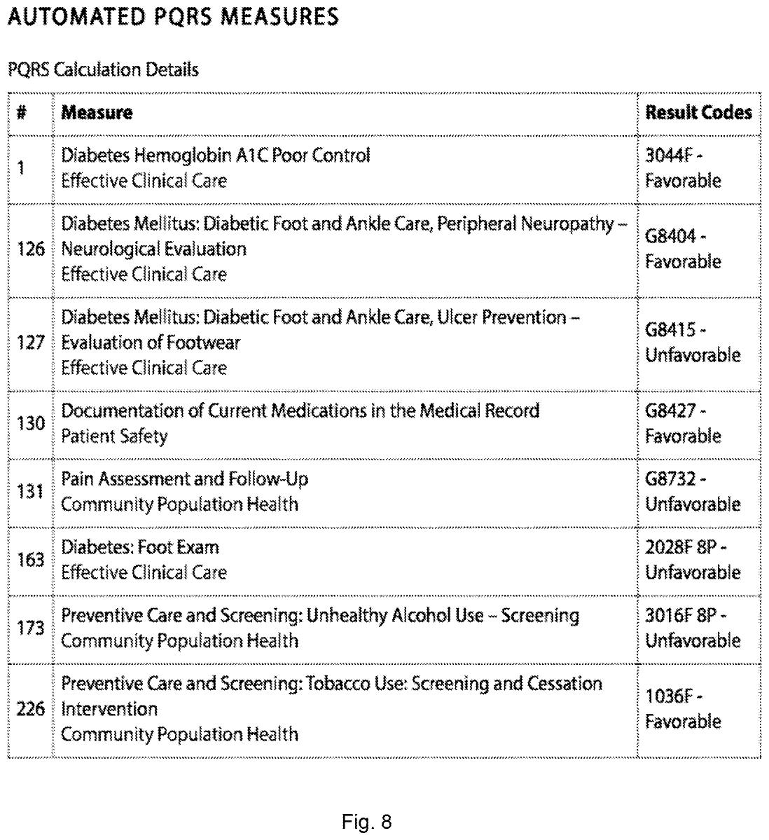

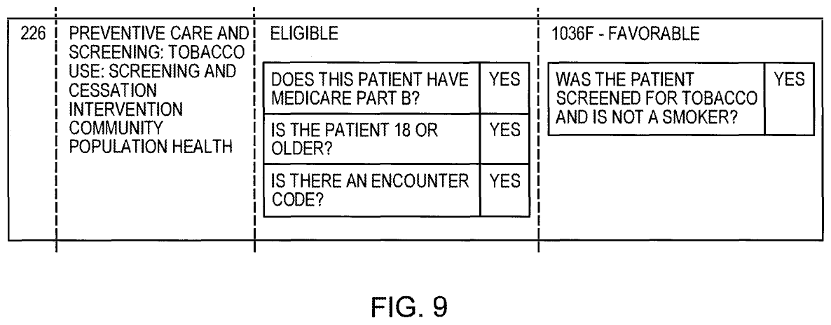

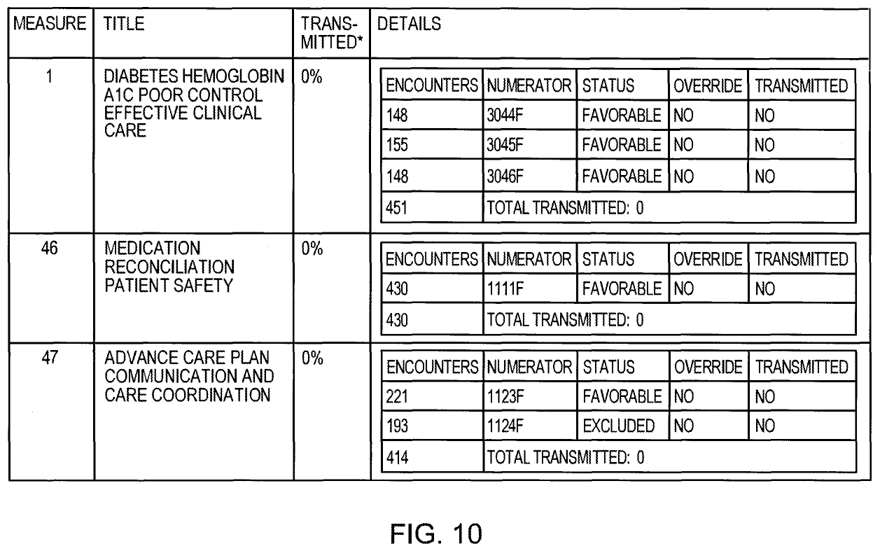

Because the calculations are made in real-time, the quality measure can be displayed as the data are entered. In one embodiment, the display screen is shown in FIG. 8. In one embodiment, each entry is an active link. Clicking on the link displays how the calculation was made. In addition to real-time display, the system in one embodiment will generate a report on a specific entry (FIG. 9), as well as one that includes a summary breakdown by measure of the CPT codes and the percentage of measures transmitted (FIG. 10). Active links in each screen allow the provider to sort and filter information by measure, date, performance, and transmission status and override the automated measure calculation from these screens. By linking down to a specific visit for a patient, the provider can see the automated results and optionally override measures.

It is to be understood that the figures and descriptions of the invention have been simplified to illustrate elements that are relevant for a clear understanding of the invention, while eliminating, for purposes of clarity, other elements. Those of ordinary skill in the art will recognize, however, that these and other elements may be desirable. However, because such elements are well known in the art, and because they do not facilitate a better understanding of the invention, a discussion of such elements is not provided herein. It should be appreciated that the figures are presented for illustrative purposes, and not as construction drawings. Omitted details and modifications or alternative embodiments are within the purview of persons of ordinary skill in the art.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are intended to be embraced therein.

What is claimed is:

1. An automated system for making quality measure submissions, the system comprising:
a data input system;
a data output system;
a database of descriptive data items;
a processor in communication with the data input system, the data output system and the database, and comprising a rules engine constructed to traverse a hierarchical tree of denominator and numerator questions, using patient input and data items from the database of descriptive data items.

2. The automated system of claim 1 wherein descriptive data items comprise SNOMED, ICD9, ICD10, RxNorm, LOINC, CPT and Metathesaurus code value pairs.

3. The automated system of claim 1 wherein descriptive data items comprise descriptive string and at least one of a code system and a specific code value.

4. The automated system of claim 1 wherein descriptive data items are linkable to other descriptive data items.

5. The automated system of claim 1 wherein the system applies a descriptive data item to a specific object input by a user.

6. The automated system of claim 5 wherein the user associates a fact with the specific object and the system assigns a descriptive data item to the fact associated with the specific object.

7. The automated system of claim 6 wherein the system determines whether the descriptive data item assigned to the fact should be assigned as metadata.

8. The automated system of claim 7 wherein the rules engine examines the facts associated with an object and applies a hierarchical tree of rules to metadata assigned to the facts to determine if the facts meet the requirements of the rules.

9. The automated system of claim 8 wherein the rules engine uses the metadata assigned to the facts to determine an outcome value of a specific rule.

10. The automated system of claim 9 wherein the rules engine associates the outcome with the object to which the fact applies.

11. The automated system of claim 9 wherein the rules engine determines an outcome value of a specific rule in response to an outcome value generated by other rules.

12. The automated system of claim 9 wherein the rules engine determines an outcome value of a specific rule in real time to output the outcome value to the user.

13. The automated system of claim 9 wherein the system generates an output report in response to all objects for which the user has associated facts.

14. An automated review system for providing a multiple-measure review of patient care comprising:
a provider input device for inputting patient data;
a patient database;
a rules engine in communication with the patient database and traversing a plurality of denominator and numerator rules, using both provider input patient data and patient data from the patient database to generate, from multiple encounters and multiple measures the review of patient care subsequent to each patient visit.

15. The system of claim 2 wherein the review is automatically provided to a PARS registry.
